(19) United States
(12) Patent Application Publication
(10) Pub. No.: US 2021/0353148 A1
(43) Pub. Date: Nov. 18, 2021

(54) SYSTEMS AND METHODS FOR SIMULTANEOUS WOUND DETECTION AND THERAPY

(71) Applicant: CHEMIMAGE CORPORATION, Pittsburgh, PA (US)

(72) Inventor: Shona STEWART, Pittsburgh, PA (US)

(21) Appl. No.: 17/320,933

(22) Filed: May 14, 2021

Related U.S. Application Data
(60) Provisional application No. 63/024,728, filed on May 14, 2020.

(51) Int. Cl. A61B 5/00; A61N 5/06

(52) U.S. Cl. CPC A61B 5/0036; A61B 5/0077; A61N 5/062; A61B 5/444; A61N 5/0616; A61B 5/445; A61N 2005/067; A61N 2005/0659; A61N 2005/0661; A61N 2005/0663; A61N 2005/0653; A61N 2005/0654; A61N 2005/0656; A61N 2005/0628

(57) ABSTRACT

Medical conditions in tissues are simultaneously imaged and treated using light within selected wavelength ranges. By treating conditions, such as wounds, lesions, and tumors, at the same time that they are imaged, the overall diagnostic and treatment time is substantially reduced.

### CROSS-REFERENCE TO RELATED APPLICATIONS

This application claims the benefit of U.S. Provisional Patent Application No. 63/024,728 filed May 14, 2020, the entirety of which is incorporated by reference herein.

### FIELD

The present disclosure relates generally to systems and methods for simultaneous medical imaging and therapy. More particularly, light of specified wavelength and intensity is applied both to generate useful images and to treat certain conditions.

### BACKGROUND

Light is used in medical imaging to non-invasively identify various structures that are of interest in medical procedures and diagnostics. For example, light of various wavelengths can be used to create hyperspectral images that identify skin lesions, tumors, and even levels of tissue oxygenation. The advantages of using light are many, including the avoidance of invasive tests like biopsies or even exploratory surgery and the simplicity by which light is generated and directed to organs and other anatomical structures.

Light is also used in medical therapies to treat conditions of skin and other organs, as well as improve psychological wellbeing. In addition to light exposure being an effective therapeutic treatment in and of itself, light can also be used to trigger the release or action of pharmaceutical compounds. Such triggered or released pharmaceutical compounds can have myriad uses, including treating the body directly or interacting with other useful compounds that treat the body.

Even so, there is a continual need for medical treatments and diagnostic tests that are efficient and minimally invasive. In particular, it would be desirable to combine the diagnostic and imaging capabilities of various light spectra with the therapeutic capabilities of the same or similar spectra.

### SUMMARY

This summary is provided to comply with 37 C.F.R. § 1.73, requiring a summary of the invention briefly indicating the nature and substance of the invention. It is submitted with the understanding that it will not be used to interpret or limit the scope or meaning of the claims.

In one embodiment, there is a method of imaging and treating a condition of a tissue, the method comprising: generating photons with a light source; directing the generated photons to a tissue, said tissue comprising the condition; interacting the generated photons with the tissue, the condition, or both the tissue and the condition in order to treat the condition, simultaneously to the interacting, collecting interacted photons that have interacted with the tissue, the condition, or both the tissue and the condition; directing the collected and interacted photons to a camera chip, generating, by way of the camera chip, an image from the collected, interacted photons.

In another embodiment, the condition is one or more of a wound, a cancer, a non-cancerous tumor, a skin lesion, or a pre-cancerous skin lesion.

In another embodiment, treating the condition is performed photodynamically.

In another embodiment, the photodynamic treatment includes application of a photosensitizing agent to one or more of the tissue or the condition.

In another embodiment, treating the condition is performed by the direct therapeutic effect of interacting the generated photons with the tissue or the condition.

In another embodiment, treating the condition is performed photodynamically by applying a photosensitizing agent to one or more of the tissue or the condition and by the direct therapeutic effect of interacting the generated photons the tissue or the condition.

In another embodiment, the generated photons have wavelengths of one or more of ultraviolet (UV), visible (VIS), near infrared (NIR), visible-near infrared (VIS-NIR), shortwave infrared (SWIR), extended shortwave infrared (eSWIR), or near infrared-extended shortwave infrared (NIR-eSWIR).

In another embodiment, the generated photons are VIS and have one or more wavelength ranges corresponding to violet, blue, cyan, green, yellow, orange, or red.

In another embodiment, the generated photons are UV.

In another embodiment, the light source comprises one or more of an incandescent lamp, a halogen lamp, a light emitting diode (LED), a chemical laser, a solid state laser, an organic light emitting diode (OLED), an electroluminescent device, a fluorescent light, a gas discharge lamp, a metal halide lamp, a xenon arc lamp, and an induction lamp.

In another embodiment, the light source is tunable.

In another embodiment, the method further comprising filtering the interacted photons.

In another embodiment, the method further comprises filtering interacted photons, and the filtering interacted photons is performed by one or more of a fixed filter, a multi-conjugate filter, and a conformal filter.

In another embodiment, the method further comprises determining the existence of the condition before generating photons, directing the generated photons, interacting the generated photons, directing the collected and interacted photons, and generating an image.

In another embodiment, determining the existence of the condition includes separate antecedent steps to the steps of generating photons, directing the generated photons, interacting the generated photons, and directing the collected and interacted photons, and the antecedent steps comprise: antecedently generating photons, antecedently collecting photons, antecedently interacting photons, and antecedently directing the photons, to thereby diagnose the condition.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

### Light Source

In an embodiment, at least one light source that generates photons that are directed to a human or animal. The light source is not limited and can be any source that is useful in providing the illumination. In an embodiment, the at least one light source may be used in concert with or attached to endoscope. Other ancillary requirements, such as power consumption, emitted spectra, packaging, thermal output, and so forth may be determined based on the particular application that the at least one light source is used. In some embodiments, the light source is a light element, which is an individual device that emits light. The light elements are not limited and may include an incandescent lamp, halogen lamp, light emitting diode (LED), chemical laser, solid state laser, organic light emitting diode (OLED), electroluminescent device, fluorescent light, gas discharge lamp, metal halide lamp, xenon arc lamp, induction lamp, or any combination of these light sources. In other embodiments, the light source is a light array, which is a grouping or assembly of more than one light element that are placed in proximity to each other.

In some embodiments, a light source has a particular wavelength that is intrinsic to the light element or to the light array. In other embodiments, the wavelength of the light sources is modified by filtering or tuning the photons that are emitted by the light source. In still other embodiments, light sources having different wavelengths are combined. The selected wavelength of a light source is not limited and can be one or more of ultraviolet (UV), visible (VIS), near infrared (NIR), visible-near infrared (VIS-NIR), shortwave infrared (SWIR), extended shortwave infrared (eSWIR), and near infrared-extended shortwave infrared (NIR-eSWIR) ranges. These correspond to wavelengths of about 180 nm to about 380 nm (UV), about 380 nm to about 720 nm (VIS), about 400 nm to about 1100 nm (VIS-NIR), about 850 nm to about 1800 nm (SWIR), about 1200 nm to about 2450 nm (eSWIR), and about 720 nm to about 2500 nm (NIR-eSWIR). The above ranges may be used alone or in combination of any of the listed ranges. Such combinations include adjacent (contiguous) ranges, overlapping ranges, and ranges that do not overlap.

Within the VIS light range, different colors of light can be employed alone or in combination. Violet light has a wavelength of about 380 to about 450 nm, blue light has a wavelength of about 450 to about 485 nm, cyan light has a wavelength of about 485 to about 500 nm, green light has a wavelength of about 500 nm to 565 nm, yellow light has a wavelength of about 565 nm to about 590 nm, orange light has a wavelength of about 590 nm to about 625 nm, and red light has a wavelength of about 625 nm to about 720 nm.

In some embodiments, the light source is a modulated light source. The choice of modulated light source and the techniques of modulating the light source are not limited. In some embodiments, the modulated light source is one or more of a filtered incandescent lamp, filtered halogen lamp, tunable LED array, tunable solid state laser array, tunable OLED array, tunable electroluminescent device, filtered fluorescent light, filtered gas discharge lamp, filtered metal halide lamp, filtered xenon arc lamp, filtered induction lamp, or any combination of these light sources. In some embodiments, tuning is accomplished by increasing or decreasing the intensity or duration at which the individual light elements are powered. Alternatively, tuning is accomplished by a fixed or tunable filter that filters light emitted by the individual light elements. In still other embodiments, the light source is not tunable. A light source that is not tunable cannot change its emitted light spectra, but it can be turned on and off by the appropriate controls.

In some embodiments, the light source comprises a quartz tungsten halogen light source. In other embodiments, the illumination source may comprise a metal halide light source, a light emitting diode (LED), a LED array having a uniform selection of emitters which emit over a constant wavelength range or a plurality of emitters which emit over a diversity of wavelength ranges, a pulsed LED, a pulsed LED array, a laser, a pulsed laser, a broadband illumination source, gas discharge light source, a fluorescent light source, an arc light source, a xenon arc lamp source, an LED light source in combination with phosphors and/or quantum dots, and the like and combinations thereof. The illumination sources are selected depending on the wavelengths of interest, and in particular whether a wavelength can not only be therapeutic, but also diagnostic in nature. Of the above, the lasers and/or LED light sources may be selected depending on the wavelengths of interest. The lasers may be gas discharge or solid state or semiconductor lasers and include helium-neon, argon, krypton, xenon ion, nitrogen, carbon monoxide, eximer, dye lasers such as stilbene, coumarin, and rhodamine, solid state or semiconductor lasers such as ruby, Nd:YAG, NdCrYAG, Nd:YLF, Nd:YVO₄, Nd:YCa₄O₄, Nd:YCa₄O(BO₃)₃, Nd:glass, Ti:sapphire, Tm:YAG, Tb:YAG, Yb doped glass, Ho:YAG, Cr:ZnSe, Ce:LiSAF, Ce:LiCAF, GaN, InGaN, AlGaInP, AlGaAs, InGaAsP, and lead salt, vertical cavity surface emitting lasers, quantum cascade laser, and hybrid silicon lasers. The illumination source may have a fixed spectral emission or may be tunable by combining sources, filtering, and/or modulating the sources and/or filters. Depending on the size, thermal output, power requirements, and so forth, the illumination source may be used directly within an a system, or remotely via optical fibers that are transparent to the desired wavelengths.

### Therapy

The light sources of the present disclosure are configured so that, when activated, the light emitted by the light sources achieves therapeutic effects in a human or animal patient. The therapeutic effects are not limited. In one embodiment, the light achieves direct therapeutic effect by interacting with body tissue. In another embodiment, the light achieves direct therapeutic effect by interacting with the body tissue and the condition. In another embodiment, the light is used in photodynamic therapy where the application of light releases a therapeutic composition, and that therapeutic composition achieves the therapeutic effect. In yet another embodiment, the light simultaneously achieves direct therapeutic effect and photodynamic effect. In this embodiment, the direct therapeutic effect can be achieved by the light interacting with the body tissue, the condition, or both the body tissue and the condition.

In some embodiments, the light has a direct therapeutic effect and is in the UV spectrum. In such configurations, the light can treat one or more of atopic dermatitis, psoriasis, vitiligo, acne vulgaris, cancer, and wounds. In other embodiments, the light has a direct therapeutic effect, and the wavelength of the light is one or more of UV, red, blue, NIR, VIS-NIR, SWIR, eSWIR, NIR-eSWIR, and full spectrum.

In still further embodiments, the light does not achieve a direct effect and is instead used to perform photodynamic therapy. During photodynamic therapy, the light is directed to a photosensitizer drug that releases therapeutically beneficial or therapeutically active compounds.

The list of conditions that respond to a treatment of the type disclosed herein is not limited and includes one or more of wounds, cancer, non-cancerous tumors, skin lesions, and precancerous skin lesions.

### Visualization

For at least a portion of the time that light is emitted from the light source in order to achieve a therapeutic effect on a human or animal patient, the light is also used for visualization. The light that is used for simultaneous visualization and therapy is directed to the organs, skin, or other body tissues that are the subject of treatment. Examples of the light that is used for simultaneous visualization and therapy includes one or more of ultraviolet (UV), visible (VIS), near infrared (NIR), visible-near infrared (VIS-NIR), shortwave infrared (SWIR), extended shortwave infrared (eSWIR), and near infrared-extended shortwave infrared (NIR-eSWIR) ranges. In one embodiment, the light is ultraviolet (UV). In another embodiment, the spectral range of the light is alternated over time. Alternating or changing the spectral range of the light can be accomplished by switching light elements on or off individually, switching sections of the light array on or off individually, or switching an entire light array on or off.

Imaging is performed by filtering and detecting photons that are reflected from the body of the human or animal patient. The techniques and devices for filtering are not limited and include any of fixed filters, multi-conjugate filters, and conformal filters. In fixed filters, the functionality of the filter cannot be changed, though the filtering can be changed by mechanically moving the filter into or out of the light path. In some embodiments, the filter is a tunable filter that comprises a multi-conjugate filter. The multi-conjugate filter is an imaging filter with serial stages along an optical path in a Solc filter configuration. In such filters, angularly distributed retarder elements of equal birefringence are stacked in each stage with a polarizer between stages.

A conformal filter can filter a broadband spectra into one or more passbands. Example conformal filters include a liquid crystal tunable filter, an acousto-optical tunable filter, a Lyot liquid crystal tunable filter, an Evans Split-Element liquid crystal tunable filter, a Solc liquid crystal tunable filter, a Ferroelectric liquid crystal tunable filter, a Fabry Perot liquid crystal tunable filter, and combinations thereof.

In an embodiment, the image is collected by a camera chip. The camera chip is not limited, but in some embodiments is selected depending on the expected spectra that is reflected from the skin, tissues, or organs of the human or animal patient. In some embodiments, the camera chip is one or more of a charge coupled device (CCD), a complementary metal oxide semiconductor (CMOS), an indium gallium arsenide (InGaAs) camera chip, a platinum silicide (PtSi) camera chip, an indium antimonide (InSb) camera chip, a mercury cadmium telluride (HgCdTe) camera chip, or a colloidal quantum dot (CQD) camera chip. In some embodiments, each or the combination of the above-listed camera chips is a focal plane array (FPA). In some embodiments, each of the above camera chips includes quantum dots to tune their bandgaps thereby altering or expanding sensitivity to different wavelengths. The visualization techniques are not limited, and include one or more of VIS, NIR, SWIR, autofluorescence, or Raman spectroscopy.

Although visualization is mentioned, the disclosure is not so limited. For example, in some embodiments, the systems and methods include the ablation of body tissue by solid state lasers or chemical lasers. The tissue can be one or more of skin or organs.

In some embodiments, the visualization is performed at the same time or with at least some overlapping time with therapy. Such embodiments might be selected, for example, in situations where it is known or likely that a patient has a condition that needs to be treated. For example, a patient with a previously diagnosed condition can be safely visualized and treated at the same time without any prior diagnostic imaging. In such instances, the systems and methods described herein for performing visualization are confirmatory and can be used to analyze the condition as treatment continues.

In other embodiments, an additional antecedent series of visualization steps are performed before therapy. Such embodiments might be selected, for example, in situations where it is not known or not likely that a patient has a condition that needs to be treated. For example, for a patient suspected but not diagnosed with a condition, antecedent visualization will be performed before any treatment is performed. In such instance, the same systems and methods described herein are used to both diagnose and treat the condition. The antecedent visualization can, in certain embodiments, include the same steps described herein for visualization, but they are performed before any simultaneous treatment and visualization is performed.

In each of the above embodiments, the disclosure further contemplates that the same system or method performs both the visualization and the therapy, thereby saving time and resources. Furthermore, in those embodiments where the patient is known or likely to have a condition, the disclosure saves even further time and resources by enabling the same system or method to both treat and monitor the patient.

### EXAMPLE

A tunable laser array light source was provided and includes two tunable laser light elements. When switched on, each laser light element produced a different wavelength. The light that was output from the light array was directed to a test tissue that contained a precancerous lesion. A photosensitizing agent was applied to the tissue. A dual polarization conformal filter was also placed to receive photons that interacted with the tissue. A CCD camera chip located on the opposite side of the conformal filter from the tissue was configured to output hyperspectral images of the tissue based on the photons that interacted with the tissue and were further modified by the dual polarization conformal filter.

During operation, the above configuration captured hyperspectral images and generated a ratiometric score image. The ratiometric score image had increased contrast in comparison to images that were not generated with the combination of the tuned light source and the dual polarization conformal filter. The increased contrast permitted easier identification of the precancerous lesion. At the same time during operation, the light that was produced by the tunable laser array light source also caused the photosensitizing agent to release therapeutically beneficial compounds. Thus, the precancerous legion was not only identified by imaging light that interacted with it, but therapeutic treatment was simultaneously performed by way of the release of the photosensitizing agent.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method of imaging and treating a condition of a tissue, the method comprising:
generating photons with a light source;
directing the generated photons to a tissue, said tissue comprising the condition;
interacting the generated photons with the tissue, the condition, or both the tissue and the condition in order to treat the condition,
simultaneously to the interacting, collecting interacted photons that have interacted with the tissue, the condition, or both the tissue and the condition;
directing the collected and interacted photons to a camera chip,
generating, by way of the camera chip, an image from the collected, interacted photons.

2. The method of claim 1, wherein the condition is one or more of a wound, a cancer, a non-cancerous tumor, a skin lesion, or a pre-cancerous skin lesion.

3. The method of claim 1, wherein treating the condition is performed photodynamically.

4. The method of claim 3, wherein the photodynamic treatment includes application of a photosensitizing agent to one or more of the tissue or the condition.

5. The method of claim 1, wherein treating the condition is performed by the direct therapeutic effect of interacting the generated photons with the tissue or the condition.

6. The method of claim 1, wherein treating the condition is performed photodynamically by applying a photosensitizing agent to one or more of the tissue or the condition and by the direct therapeutic effect of interacting the generated photons the tissue or the condition.

7. The method of claim 1, wherein the generated photons have wavelengths of one or more of ultraviolet (UV), visible (VIS), near infrared (NIR), visible-near infrared (VIS-NIR), shortwave infrared (SWIR), extended shortwave infrared (eSWIR), or near infrared-extended shortwave infrared (NIR-eSWIR).

8. The method of claim 7, wherein the generated photons are VIS and have one or more wavelength ranges corresponding to violet, blue, cyan, green, yellow, orange, or red.

9. The method of claim 7, wherein the generated photons are UV.

10. The method of claim 1, wherein the light source comprises one or more of an incandescent lamp, a halogen lamp, a light emitting diode (LED), a chemical laser, a solid state laser, an organic light emitting diode (OLED), an electroluminescent device, a fluorescent light, a gas discharge lamp, a metal halide lamp, a xenon arc lamp, and an induction lamp.

11. The method of claim 1, wherein the light source is tunable.

12. The method of claim 1, further comprising filtering the interacted photons.

13. The method of claim 12, wherein the filtering is performed by one or more of a fixed filter, a multi-conjugate filter, and a conformal filter.

14. The method of claim 1, further comprising determining the existence of the condition before generating photons, directing the generated photons, interacting the generated photons, directing the collected and interacted photons, and generating an image.

15. The method of claim 14, wherein the determining the existence of the condition includes separate antecedent steps to the steps of generating photons, directing the generated photons, interacting the generated photons, and directing the collected and interacted photons, and the antecedent steps comprise:
antecedently generating photons,
antecedently collecting photons,
antecedently interacting photons, and
antecedently directing the photons,
to thereby diagnose the condition.
